(19) United States
(12) Patent Application Publication
(10) Pub. No.: US 2008/0044823 A1
(43) Pub. Date: Feb. 21, 2008

(54) Common module profiling of genes

(75) Inventors: Merridee Ann Wouters, Lindfield (AU); Richard Anthony George, Darlinghurst (AU)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Victor Chang Cardiac Research Institute Limited

(21) Appl. No.: 11/504,914

(22) Filed: Aug. 15, 2006

(51) Int. Cl. C12Q 1/68; G06F 19/00

(52) U.S. Cl. 435/6; 702/20

(57) ABSTRACT

A system for profiling a genomic sequence comprising assigning modules to a genome, wherein each module has a defined sequence characteristic and the genome is divided into modules; assigning a value or weight to a module for a given profile, wherein the presence of one or more modules in a genomic sequence contributes to the profile of the genomic sequence relative to its value or weight; analysing a genomic sequence to identify modules present; and assigning a profile to the genomic sequence based on the presence of the modules and their respective value or weight.

### FIELD OF THE INVENTION

The invention relates to systems for profiling genomic sequences.

### DESCRIPTION OF THE RELATED ART

The identification of genes responsible for human disease is useful to gain an understanding of disease mechanisms and is essential in the development of diagnostics and therapeutics. Linkage analysis of disease inheritance patterns is a successful procedure to associate a disease with a specific genomic region. Unfortunately, isolating the disease-causing gene(s) can be difficult: genomic regions are often large, containing hundreds of candidate genes, making experimental methods time consuming and expensive. Furthermore, searches for single nucleotide polymorphisms (SNPs) in the genomes of individual patients from clinical studies will produce a large number of potential gene candidates. These high-throughput analyses will require computational approaches to identify good candidates for further study.

The completion of the human genome sequencing project has permitted the development of new genome-scale bioinformatics approaches to understand disease. While some progress has been made in candidate gene prediction, these systems can, at best, only claim modest pruning of the genes in a disease interval and result in false negatives around 50% of the time.

Previous candidate gene prediction systems have largely been based on keyword similarity to known disease genes. For example, the G2D system is based on biomedical literature searches and associates pathological conditions with gene ontology (GO) terms. Candidate genes are then identified by homology to GO-annotated and disease-associated genes. The method POCUS finds candidate genes by identifying an enrichment of GO-keywords, shared InterPro domains and expression profiles among a given set of susceptibility loci relative to the genome at large. The method by Tiffin et al (Tiffin N, Kelso J F, Powell A R, Pan H, Bajic V B, Hide W A. (2005) Integration of text- and data-mining using ontologies successfully selects disease gene candidates. Nucleic Acids Res. 33, 1544-52) selects candidates according to their expression profiles within tissues associated with disease, and relationships between clinical and molecular data are identified using the eVOC anatomy ontology. The recent method SUSPECTS again compares GO, InterPro and expression libraries of putative disease genes with those known to be involved in the same disease. Similarly, GeneSeeker integrates keyword data based on mapping, expression and phenotypic databases from human and mouse studies. The method by Freudenberg and Propping (Freudenberg J, Propping P. (2002) A similarity-based method for genome-wide prediction of disease-relevant human genes. Bioinformatics., 18 S2, S110-5) is based on a measure of phenotypic similarity between diseases and produces clusters of disease genes using keywords derived from OMIM (Hamosh A, Scott A F, Amberger J, Bocchini C, Valle D, McKusick V A. (2002) Online Mendelian Inheritance in Man (OMIM), a knowledgebase of human genes and genomic disorders. Nucleic Acids Res., 30, 52-5). Recently, Franke et al 2006 (Franke L, Bakel H, Fokkens L, de Jong E D, Egmont-Petersen M, Wijmenga C. (2006) Reconstruction of a functional human gene network, with an application for prioritizing positional candidate genes. Am J Hum Genet. 78, 1011-25) developed a system based on predicted protein-protein interactions (PPIs), whereby disease genes are identified through common interactions to proteins in multiple disease intervals that have common phenotypes.

Some of these methods have been incorporated into a consensus approach that has been applied to select candidates for the complex diseases type 2 diabetes and obesity. Using a combination of methods appears to be effective for ranking candidate disease genes.

The present inventors have developed a computational system (termed ‘Common Module Profiling’ (CMP)) to predict profiles such as candidate disease genes within disease loci. These predicted disease genes, and their biochemical pathways, may constitute potential drug targets for the treatment of disease.

### SUMMARY OF THE INVENTION

In a first aspect, the present invention provides a system for profiling a genomic sequence comprising:

(a) assigning modules to a genome, wherein each module has a defined sequence characteristic and the genome is divided into modules;
(b) assigning a value or weight to a module for a given profile, wherein the presence of one or more modules in a genomic sequence contributes to the profile of the genomic sequence relative to its value or weight;
(c) analysing a genomic sequence to identify modules present; and
(d) assigning a profile to the genomic sequence based on the presence of the modules and their respective value or weight.

Preferably, the genomic sequence is an amino acid sequence of a protein and each module is a universal re-occurring unit found in protein sequences.

Preferably, the genome forms the encoding region and the encoding region is divided into different modules.

In a second aspect, the present invention provides a system for profiling an amino acid sequence to identify an associated profile, the system comprising:

(a) assigning modules to the protein coding region of a genome to divide the genome into modules, wherein each module has a defined amino acid characteristic;
(b) assigning a value or weight to a module for a given profile, wherein the presence of one or more modules in an amino acid sequence contributes to the profile of the sequence relatively to its value or weight;
(c) analysing an amino acid sequence to identify modules present; and
(d) assigning a profile to the amino acid sequence based on the presence of the modules and their respective value or weight.

The profile maybe any useful information such as a gene or loci associated with a phenotype, disease, drug-binding characteristic, trait associated to pharmacogenomics, associated interacting genes, association with a phenotype, associated or interacting modules, or the module with a particular disease or phenotype, or associated biochemical pathways, or associated modules within biochemical pathways or interacting models with profiles with characteristics described herein.

In a preferred embodiment, the phenotype is a disease or a quantitative trait locus (QTL).

In another preferred embodiment, the profile is an association with a disease.

In another preferred embodiment, the profile is a drug-binding characteristic.

In one preferred embodiment, a given value or weight of a module assigned to a profile is obtained by identifying modules associated with a given phenotype (directly or indirectly through pathways or complexes) and assigning a score based on the similarity of a module to modules associated with a specific phenotype.

In another preferred embodiment, a given value or weight of a module assigned to a profile is obtained by identifying enrichment of those modules in loci (genomic regions) known to be associated with the phenotype. For example, this can be carried out by identification of overrepresentation of particular modules in loci associated with the phenotype and score the degree of overrepresentation.

The present inventors have carried detailed analysis of genomic regions using proprietary software that can assign a value or weight to a module for a given profile. The present invention can thus identify modules in genomic sequences wherein each module has a defined sequence characteristic, associate profiles with the modules, and assign profiles to genomic sequences from the values or weights of the modules present.

For a given profile, typically a module is assigned a value or weight according to its presence in sequences associated with the profile.

In a third aspect, the present invention provides a system in computer readable form containing modules with defined amino acid characteristics wherein each module having an assigned value or weight for one or more profiles.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of each claim of this specification.

In order that the present invention may be more clearly understood, preferred embodiments will be described with reference to the following drawings and examples.

### BRIEF DESCRIPTION OF THE DRAWINGS

FIG. 1 shows sensitivity (continuous line) and proportion of predicted genes that are actually disease genes (dashed line) for OPHID (diamond), OPHIDh (circle), OPHIDlit+(triangle) and OPHIDlit−(square) at three levels of interactions (Distance). Results are shown for the 100 interval size only.

FIG. 2 shows performance of PPI data from a) OPHID, b) OPHIDh, c) OPHIDlit+ and d) OPHIDlit−. Results are shown for three levels of interaction using the shortest path length to a disease gene (Distance). Black diamonds represent the number of disease genes found. The number of non-disease genes returned at the 50-gene interval (square), 100-gene interval (triangle) and 150-gene interval (×). The number of disease genes returned by random selection at the 50-gene interval (*), 100-gene interval (circle) and 150-gene interval (+).

FIG. 3 shows CMP performance at different thresholds for the 100 gene interval size, based on ten diseases. Black bars represent the percentage of disease genes found. Gray bars represent the proportion of predictions that are actually disease genes.

FIG. 4 shows candidate gene enrichment for the 50 (a), 100 (b) and 150 (c) gene interval size. Black diamonds represent enrichment of data sets using the combined methods. Gray squares represent enrichment of data using random selection. Disease genes are listed alphabetically from left to right on the x-axis, as in Table 1.

FIG. 5 shows combined prediction success. a) Correct predictions based on known disease genes. b) Correct predictions based on multiple intervals c) Combined CPS and CMP predictions for familial hypertrophic cardiomyopathy (cfh). Disease genes are represented by their ENTREZ-name. Gene-linking lines are predictions by CPS and CMP. PRKAG2 and TPM1 where found using PPI data at a distance of three, all others found by PPI data were found at a distance of one.

### DETAILED DESCRIPTION OF THE INVENTION

A combined bioinformatics approach that encompasses methods of sequence comparison and protein pathway and interaction data analysis has been developed by the present inventors. Two methods can be combined for the automated prediction of disease genes within known disease intervals.

The first, Common Pathway Scanning (CPS), is based on the assumption that common phenotypes are generally associated with disruption in proteins that participate in the same complex or pathway. Recently, Gandhi et al 2006 (Gandhi T K, Zhong J, Mathivanan S, Karthick L, Chandrika K N, Mohan S S, Sharma S, Pinkert S, Nagaraju S, Periaswamy B (2006) Analysis of the human protein interactome and comparison with yeast, worm and fiy interaction datasets. Nature Genet. 38, 285-93) showed that disease-genes preferentially interact with other disease-causing genes. There are currently over 200 biological pathway and network resources available. The present inventors have utilised data from BioCarta (www.biocarta.com), KEGG and OPHID, the most comprehensive databases of their type. BioCarta and KEGG are chiefly pathway databases with BioCarta specialising in signalling pathways and KEGG in metabolic pathways. OPHID is a secondary PPI database containing literature-derived interaction data from BIND, MINT and HPRD, as well as data from recent high-throughput experimentation. OPHID also contains transferred interactions from orthologous proteins in model organisms.

The second method and useful part of the present invention, Common Module Profiling (CMP), is based on the principle that candidate genes may have similar functions to disease genes that have already been determined. This is analogous in concept to methods using functional annotations, but many human proteins lack annotation and, therefore, similarities would be missed when comparing keywords alone. For example, only 10,000 human proteins, approximately 25% of the human proteome, have manually curated GO-terms.

CMP uses a domain-based (modules) comparative sequence analysis to identify those proteins with potential functional-similarity. Domain based sequence comparison searches have been shown to be more accurate than full-sequence searches as commonly applied in BLAST or PSI-BLAST database searches. Unlike the keyword systems, CMP calculates a measure of domain-based similarity to known disease genes rather than a binary comparison.

Both methods use two sources of input for disease-gene prediction: firstly, known disease genes are used to predict novel disease genes in intervals of the same disease-phenotype and secondly, without knowledge of the disease-genes, all the genes in the multiple intervals of the same phenotype are used to find protein relationships to predict candidate disease genes.

Linkage analysis is a successful procedure to associate disease with specific genomic regions. Unfortunately, these regions are often large, containing hundreds of genes, which make experimental methods employed to identify the disease gene arduous and expensive. It is important, therefore, to prioritise likely disease genes and discount those that are unlikely to be involved in the disease. We present a computational approach to prioritise candidate disease genes for further experimental study. Starting with a disease interval, two algorithms can be applied: Common Module Profiling (CMP) and Common Pathway Scanning (CPS), which are computational versions of traditional approaches to candidate selection. CPS applies network data derived from protein-protein interaction and pathway databases to identify relationships to known disease genes. CPS is based on the assumption that common phenotypes are associated with dysfunction in proteins that participate in the same complex or pathway. CMP identifies likely candidates using a domain-dependent sequence similarity approach, based on the hypothesis that disruption of genes of similar function will lead to the same phenotype. Both algorithms use two forms of input data: known disease genes or multiple disease loci. When using known disease genes as input, our combined methods have a sensitivity of 0.518 and a specificity of 0.966 and reduced the candidate list by 13-fold. Using multiple loci, our methods successfully identify disease genes for all benchmark diseases with a sensitivity of 0.835 and a specificity of 0.626. Our combined approach prioritizes good candidates and will accelerate the disease gene discovery process.

### Materials and Methods

### Annotation Pipeline

All biological data was combined into a relational database. Human disease gene information was extracted from the OMIM database and lists of genes flanking the disease genes were obtained from EntrezGene (build 35). Protein sequence data was taken from GenBank and complete protein domain annotation was performed on all protein sequences using Pfam Hidden Markov Models (version 18). Finally, all genes were mapped to the latest pathway and PPI data downloaded from BioCarta, KEGG and OPHID.

### Common Pathway Scanning

Potential disease genes were predicted by identifying all proteins within a disease interval that are part of a pathway, described in BioCarta and KEGG. PPI data from OPHID was used to identify novel disease genes by identifying the interaction partners of known disease genes in a disease interval. Three levels of interactions are tested for potential disease genes, based on the shortest path length to a disease gene. When CPS is applied across multiple intervals, i.e. in the absence of known disease genes, all interaction partners and pathways associated with the genes in each interval are compared. Disease genes are predicted by identifying common pathways or interaction partners between the intervals.

### Common Module Profiling

CMP compares the Pfam-domain content of each protein within a disease interval to identify putative disease genes. Different calculations are performed depending on whether CMP uses known disease genes or multiple intervals as input.

When known disease genes are used as input, a protein (candidate) observed to have disease-like domains is assigned a score (S) based on the similarity between the protein's domains (j) and the domains (i) in the known disease gene (dg) using SSEARCH bit scores(s). SSEARCH is an implementation of the Smith and Waterman local alignment algorithm. Scores were normalised by matching the equivalent region of the disease gene against itself on a domain by domain basis (equation 1).

$$S = \frac{{\sum }_{i} max ( s ( {dg}_{i} , {candidate}_{j} ) )}{{\sum }_{i} s ( {dg}_{i} , {dg}_{i} )} j = 1 … N \qquad ( 1 )$$

Where a protein has multiple domains of the same type, the highest scoring matching domain is used.

When CMP is used across multiple intervals, a census of all domains in every interval associated with the disease is taken. A similarity score based on the numerator of equation 1 is calculated as well as two calculations of statistical significance. In the first calculation of significance, domains in a sequence are assumed to be completely uncorrelated, this represents an upper limit of significance. The expected (eₐ) number of genes containing those domains is calculated by:

$${e}_{a} = mnf {\prod }_{i} {P}_{i} \qquad ( 2 )$$

where m is the number of intervals containing the domains of interest; n is the number of genes in the interval; and f is a form factor, related to the average number of domains per gene. The probability of encountering domain i is given by:

$${P}_{i} = \frac{{N}_{i}}{N} \qquad ( 3 )$$

here N is all domain types. These numbers are determined from a census of all domains across the genome. For the second calculation of significance, domains are assumed to be completely correlated, this represents a lower limit of significance. The expectation (e_{b}) is based on the prevalence of the rarest domain:

e_{b}=mnf·min (Pi) (4)

Two X² tests (X²c and X²b) are then calculated in the usual manner using the two expectation values at a significance of 0.995. Clusters of genes containing the same domains are then ranked according to the two alternative values

### Benchmarking

The prediction algorithms were validated using data from previously determined disease intervals where at least three disease genes have been identified. The disease genes are used to generate pseudo-intervals. Three pseudo-interval sizes are used that encompass 50, 100 and 150 genes around the known disease genes.

When the disease genes were used as the input, the predictive power of each algorithm was tested on each disease gene using leave-one-out cross validation. In this method, one of the disease genes was disregarded and the remaining known disease genes were used to identify the omitted disease gene in its pseudo-interval. If there is not information about the disease genes, all genes in the intervals sharing a phenotype were used to identify common relationships.

Several measures of predictive power were used: sensitivity, the probability of finding a disease gene among disease genes (TP/(TP+FN)); and specificity, the probability of not finding a disease gene among non-disease genes (TN/(TN+FP)); where TP is the number of true positives, TN is the number of true negatives, FP is the number of false positives and FN is the number of false negatives. An enrichment ratio (ER) was also calculated for each disease from the proportion of disease genes predicted by the methods divided by the proportion of disease genes within the disease intervals (equation 5).

$$ER = \frac{TP / ( TP / FP )}{( \sum disease genes / \sum all genes )} \qquad ( 5 )$$

CPS and CMP predictions were compared with a random selection of candidate genes within a disease interval. The number of random assignments made was based on the number of predictions made by each method. Random selections were performed 1000 times for each disease, from which an average number of correctly identified disease genes is calculated.

### Results

Table 1 shows the results of candidate gene prediction for each of the two methods on the 29 diseases as used by Turner et al. (Turner F S, Clutterbuck D R, Semple C A. (2003) POCUS: mining genomic sequence annotation to predict disease genes. Genome Biol., 4, R75) in their analysis of POCUS. Complete lists of all disease genes and pseudo-intervals used for benchmarking are available at our web site www.pathologene.org. The present invention made predictions for all 29 diseases in each of the 50, 100 and 150-gene intervals and correctly predict a disease gene in 20 of the 29 diseases, finding 88 of the total 170 disease genes. In comparison, POCUS made candidate predictions for eight of the 29 diseases for interval sizes averaging 94 genes and only five of the diseases had a disease gene correctly predicted.

TABLE 1
Number of correctly predicted disease genes by each method using known disease genes.

| | Known | Successful Automated Predictions | | | | | | | | | | |
|---|---|---|---|---|---|---|---|---|---|---|---|---|
| | Disease | | CPS | | | | | | | Random | | |
| Disease | Genes | CMP | BioCarta | KEGG | OPHID | OPHIDh | OPHIDlit+ | OPHIDlit− | Total | 50 | 100 | 150 |
| aan | 4 | 0 | 0 | 0 | 3 | 3 | 3 | 2 | 3 | 0.1 | 0.1 | 0.1 |
| alz | 8 | 2 | 3 | 6 | 5 | 5 | 5 | 3 | 6 | 0.3 | 0.2 | 0.2 |
| aml | 4 | 0 | 0 | 0 | 0 | 0 | 0 | 0 | 0 | 0.2 | 0.2 | 0.2 |
| bb | 4 | 0 | 0 | 0 | 0 | 0 | 0 | 0 | 0 | 0.0 | 0.0 | 0.0 |
| bc | 9 | 0 | 4 | 0 | 6 | 6 | 6 | 0 | 6 | 0.5 | 0.5 | 0.5 |
| bcc | 4 | 1 | 1 | 2 | 3 | 3 | 3 | 0 | 3 | 0.1 | 0.0 | 0.1 |
| cchn | 6 | 5 | 0 | 0 | 5 | 4 | 4 | 4 | 5 | 0.4 | 0.3 | 0.3 |
| cf | 5 | 0 | 2 | 2 | 0 | 0 | 0 | 0 | 2 | 0.2 | 0.2 | 0.2 |
| cfh | 12 | 5 | 0 | 4 | 4 | 4 | 4 | 0 | 9 | 1.0 | 0.7 | 0.8 |
| cmt | 5 | 0 | 0 | 0 | 2 | 2 | 2 | 0 | 2 | 0.2 | 0.2 | 0.2 |
| ebl | 5 | 3 | 0 | 5 | 5 | 5 | 5 | 0 | 5 | 0.2 | 0.1 | 0.1 |
| ed | 7 | 5 | 0 | 2 | 0 | 0 | 0 | 0 | 5 | 0.4 | 0.3 | 0.2 |
| fap | 4 | 0 | 0 | 3 | 0 | 0 | 0 | 0 | 3 | 0.2 | 0.2 | 0.1 |
| gc | 5 | 0 | 2 | 3 | 0 | 0 | 0 | 0 | 4 | 0.3 | 0.2 | 0.2 |
| h | 5 | 0 | 0 | 0 | 0 | 0 | 0 | 0 | 0 | 0.1 | 0.2 | 0.2 |
| ibd | 5 | 0 | 2 | 3 | 4 | 4 | 4 | 2 | 4 | 0.4 | 0.3 | 0.3 |
| joag | 4 | 0 | 0 | 0 | 0 | 0 | 0 | 0 | 0 | 0.1 | 0.1 | 0.1 |
| lca | 6 | 0 | 0 | 0 | 0 | 0 | 0 | 0 | 0 | 0.1 | 0.1 | 0.1 |
| lhscr | 5 | 0 | 0 | 2 | 2 | 2 | 2 | 0 | 4 | 0.2 | 0.3 | 0.3 |
| md | 6 | 2 | 0 | 0 | 2 | 2 | 2 | 0 | 3 | 0.1 | 0.1 | 0.1 |
| mf | 4 | 0 | 0 | 0 | 0 | 0 | 0 | 0 | 0 | 0.2 | 0.2 | 0.2 |
| mody | 6 | 2 | 0 | 0 | 4 | 4 | 4 | 2 | 5 | 0.3 | 0.3 | 0.3 |
| niddm | 8 | 4 | 2 | 0 | 2 | 2 | 2 | 2 | 5 | 0.6 | 0.4 | 0.3 |
| oc | 4 | 0 | 0 | 4 | 2 | 2 | 2 | 2 | 4 | 0.3 | 0.3 | 0.3 |
| pc | 6 | 0 | 0 | 0 | 0 | 0 | 0 | 0 | 0 | 0.1 | 0.1 | 0.2 |
| pd | 3 | 0 | 0 | 3 | 2 | 2 | 2 | 0 | 3 | 0.1 | 0.0 | 0.0 |
| rp | 10 | 0 | 0 | 0 | 0 | 0 | 0 | 0 | 0 | 0.2 | 0.2 | 0.2 |
| sle | 3 | 0 | 0 | 0 | 0 | 0 | 0 | 0 | 0 | 0.2 | 0.1 | 0.2 |
| tcp | 13 | 3 | 0 | 2 | 4 | 4 | 4 | 0 | 7 | 0.9 | 0.8 | 0.8 |
| Total | 170 | 32 | 16 | 41 | 55 | 54 | 54 | 17 | 88 | 8.0 | 6.6 | 6.7 |

CMP results are based on a cut-off threshold of 0.1. CPS-interactions go to the 1 st level of interaction only. CPS-OHPID contains all PPI data from OPHID. CPS-OPHIDh contains human data only. CPS-OPHIDlit+ contains data from literature databases only. CPS-OPHIDlit− does not contain PPI data from literature databases. Random is calculated on total predictions for the 50, 100 and 150 interval sizes. Disease abbreviations: aan, adrenoleukodystrophy, autosomal neonatal; alz, Alzheimer disease; aml, acute myeloid leukemia; bb, Bardet-Biedl syndrome; bc, breast cancer; bcc, basal cell carcinoma; cchn, colorectal cancer, hereditary nonpolyposis; cf, cystic fibrosis; cfh, cardiomyopathy, familial hypertrophic; cmt, Charcot-Marie-Tooth disease; ebl, epidermolysis bullosa letalis; ed, epiphyseal dysplasia, multiple types 1-5; fap, familial adenomatous polyposis; gc, gastric cancer; h, hypertension; ibd, inflammatory bowel disease; joag, juvenile-onset primary open angle glaucoma; lca, Leber congenital amaurosis; lhscr, long-segment Hirschsprung disease; md, muscular dystrophy, limb-girdle; mf, familial meningioma; mody, maturity-onset diabetes of the young; niddm, type 2 diabetes mellitus; oc, ovarian carcinom; pc, prostate cancer; pd, Parkinson disease; rp, retinitis pigmentosa; sle, systemic lupus erythematosus; tcp, thyroid carcinoma, papillary.

### CPS Benchmark Performance Using Known Disease Genes

CPS identifies novel disease genes by finding proteins that are linked with the product of a known disease gene in the pathway and PPI databases. Results for CPS are divided into three datasets: pathway data from BioCarta, pathway data from KEGG and PPI data from OPHID. KEGG pathway data correctly predicts 41 disease genes in 13 diseases. For the 100-gene interval size, the probability of finding a disease gene (sensitivity) using KEGG data is 0.257, and the probability of not finding a disease gene among non-disease genes (specificity) by KEGG is 0.981. Overall data enrichment is 12-fold for the 100-gene interval size.

BioCarta pathway data identifies 16 disease genes in seven diseases. BioCarta has a sensitivity of 0.152, a specificity of 0.992 and an enrichment of 16-fold for the 100-gene interval size. The complementary nature of these pathway databases is demonstrated by their unique results. BioCarta finds disease genes for two diseases, type 2 diabetes mellitus and breast cancer, where KEGG fails. KEGG finds disease genes for eight diseases where BioCarta fails.

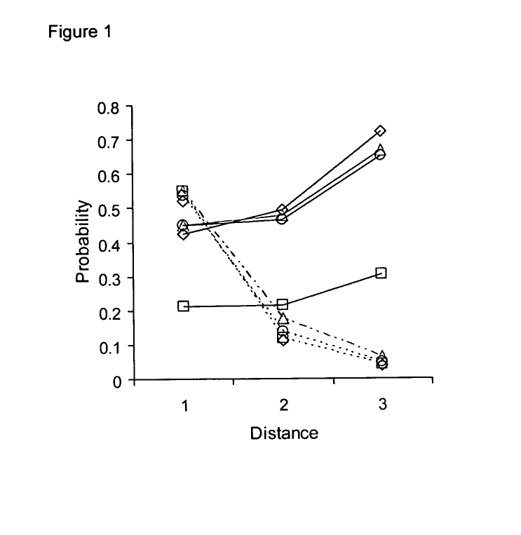

The OPHID PPI dataset contains 48,321 interactions for 10,666 proteins representing 13% of the estimated complete human-interactome. Overall, OPHID has a sensitivity of 0.423, a specificity of 0.996 and an enrichment of 50-fold at the 100-gene interval size. These results are much better than the pathway data, but the success of prediction using PPI data might be influenced by PPI data derived from literature associations of well studied diseases. In an attempt to remove bias from literature PPIs and to assess the usefulness of orthology data, OPHID is further split into several overlapping sets: human-only data, i.e. the data does not contain transferred orthologous interactions (OPHIDh); PPI data derived from literature searches only, i.e. data from the BIND, HPRD and MINT databases (OPHIDlit+); and all PPIs except those from the literature databases (OPHIDlit−). The difference between OPHID and OPHIDh predictions is small: OPHID finds one more disease gene than OPHIDh, but with slightly more false positives. FIG. 1 shows the sensitivities for each of the datasets compared with the proportion of correct predictions at increasing path lengths for the 100-gene interval size. At the first level of interactions the majority of correct predictions, 54, is found using the OPHIDlit+ set, with a sensitivity of 0.45 and specificity of 0.996. The non-literature PPIs find 17 disease genes, with a sensitivity of 0.213 and a specificity of 0.996. While the probability of finding a disease gene is lower in the non-literature set, overall data-enrichment is the same, 53-fold, and the proportion of correct predictions is the same, 0.55. Therefore, it is the larger coverage of the literature data that gives it the advantage over the non-literature set and suggests that the experimental data and orthology data held in the OPHIDlit− set is of equal quality to the literature assignments.

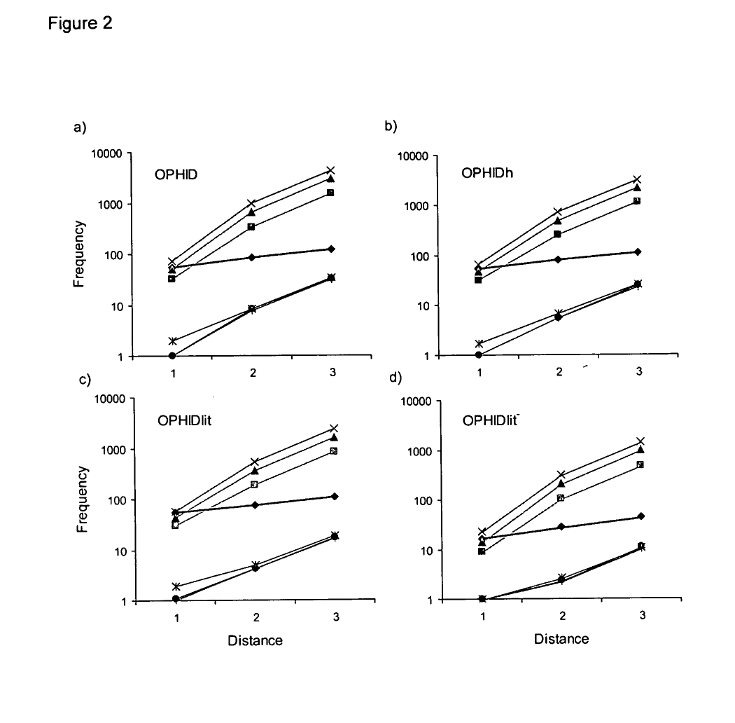

FIG. 2 shows the number of false positives returned by the interaction data at increasing path lengths up to a distance of three interactions from the known disease genes. As the shortest path length increases the sensitivity improves but the number of false positives increases exponentially reducing specificity. At a distance of two interactions, the full OPHID set finds 84 disease genes with a sensitivity of 0.494, a specificity of 0.96 and an enrichment of 11-fold. Increasing the distance to three interactions, finds 123 disease genes, with a high sensitivity of 0.723, but a smaller specificity of 0.816 and a poor four-fold enrichment.

Combining the results from the full OPHID set (where the shortest path length is one) with the results from BioCarta and KEGG, CPS makes predictions for 28 diseases and identifies 78 disease genes. Overall CPS performance has a sensitivity of 0.47 with a specificity of 0.977 and an enrichment of 17-fold at the 100-gene interval size. Less than 0.6% of proteins rejected will be disease genes.

### CPS Benchmark Performance Using Multiple Intervals

When multiple loci are used as the input to CPS, 100 disease genes were correctly identified in the 100-gene intervals. While sensitivity was high 0.588, more false positives were predicted compared to input from known disease genes. This reduced specificity to 0.844 and the enrichment ratio to 3.7-fold. The pathway and PPI data complement each other: CPS using pathway data alone finds 28 disease genes that are missed by the PPI data. Conversely, CPS using PPI data alone finds 33 disease genes that the pathway data misses and together they find the same 39 disease genes. In the absence of known disease genes, the use of network data on multiple disease-loci is a powerful approach to identify disease genes. Table 2 shows the results for each of the individual methods.

TABLE 2
Multiple loci benchmark results.

| | 50 | | | 100 | | | 150 | | |
|---|---|---|---|---|---|---|---|---|---|
| Method | Sens. | Spec. | ER | Sens. | Spec. | ER | Sens. | Spec. | ER |
| CPS-Pathway | 0.353 | 0.903 | 3.4 | 0.394 | 0.886 | 3.4 | 0.406 | 0.875 | 3.2 |
| CPS-PPI | 0.394 | 0.953 | 7.3 | 0.424 | 0.934 | 6.1 | 0.471 | 0.919 | 5.6 |
| CPS | 0.541 | 0.873 | 4.0 | 0.588 | 0.844 | 3.7 | 0.624 | 0.824 | 3.5 |
| CMP (X²a | 0.165 | 0.953 | 3.3 | 0.188 | 0.941 | 3.1 | 0.229 | 0.929 | 3.2 |
| multi) | | | | | | | | | |
| CMP (X²a all) | 0.459 | 0.769 | 1.9 | 0.553 | 0.715 | 1.9 | 0.588 | 0.688 | 1.9 |
| CMP (X²b | 0.159 | 0.954 | 3.2 | 0.176 | 0.944 | 3.1 | 0.218 | 0.935 | 3.3 |
| multi) | | | | | | | | | |
| CMP (X²b all) | 0.459 | 0.770 | 2.0 | 0.553 | 0.716 | 1.9 | 0.582 | 0.690 | 1.9 |
| CPS-CMP (X²a | 0.741 | 0.692 | 2.3 | 0.835 | 0.626 | 2.2 | 0.865 | 0.592 | 2.1 |
| all) | | | | | | | | | |

Abbreviations:
Sens., sensitivity;
Spec., specificity;
ER, Enrichment Ratio;
X²a, significance based on the assumption that domains in a gene are uncorrelated;
X²b, significance based on the assumption that domains in a gene are correlated;
multi, genes that contain multiple Pfam-domains only;
all, genes that contain at least one Pfam domain.
All X² tests are at a significance level of 0.995.

### CMP Benchmark Performance from Known Disease Genes

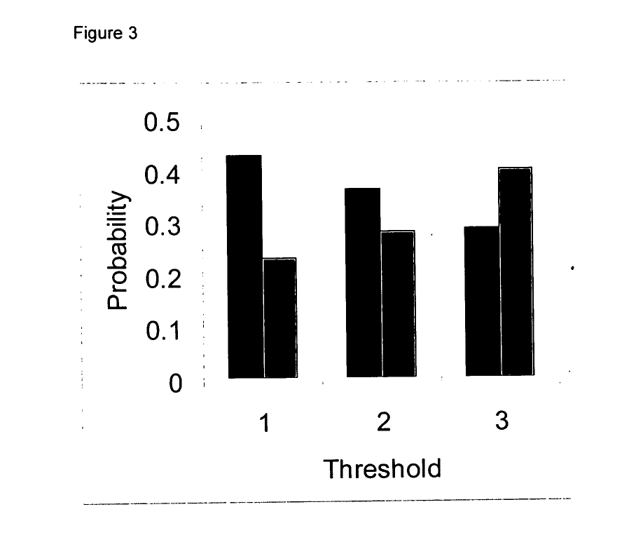

CMP identifies disease genes using domain-based comparative sequence analysis. This was achieved by first using Pfam Hidden Markov Models to annotate the domain content of known disease genes. Putative disease genes were then identified based on a shared domain content with the known disease genes. FIG. 3 shows the performance of CMP at three score thresholds for the 100-gene gene interval. The ratio of true positives to false positives was best at a threshold of 0.4. However, at a threshold of 0.1, CMP found more disease genes and sensitivity was at its best. At this threshold, 7.5%, 11.6% and 18.5% of predictions are disease-causing genes for the 50, 100 and 150-gene intervals, respectively. Less than 0.8% of proteins rejected will be disease genes.

Independently, CMP correctly predicts 32 disease genes for 10 diseases at a score threshold of 0.1 and has a sensitivity of 0.2 and a specificity of 0.98 for each interval size. Overall enrichment for all diseases was 11-fold at the 100-gene interval size.

### CMP Benchmark Performance Using Multiple Intervals

When multiple loci were used as the input to CMP, a census of the domain content of all genes in the specified loci was taken. The numbers of genes with a specific domain content were compared with the expected number of genes based on the prevalence of those domains in the genome (see Methods). Clusters of genes with similar domain content were ranked based on two estimates of the significance: the first assumed that the domain content of the cluster is completely uncorrelated and is an upper estimate of the significance (X²a); the second assumed the domains are highly correlated and the prevalence is determined by the rarest domain (X²b). These two values are the same for single domain proteins.

Comparison of the CMP results are shown in Table 2. Results have been split into subgroups: those that contain multiple Pfam domains (multi) and those that contain at least one Pfam domain (all). Sensitivity is low for the multidomain method because disease genes with zero or one Pfam domain are included in the false negatives. However, the specificity was very high indicating that if the target disease genes were multiple domain proteins, the method is very effective.

The 36 disease genes potentially identifiable by CMP, based on their domain similarity, can be divided into 16 clusters, containing two or more disease genes. Of these genes, 32 were identified by CMP using known disease genes as a starting point, while four fell below the 0.1 threshold similarity. Using multiple intervals as input, two clusters containing four genes were not found as determined by significance. For example, genes RET and NTRK1 involved in thyroid carcinoma have a protein kinase domain in common, but protein kinase domains are very common in the genome and thus lowered the significance of the shared domain.

Of the 14 successfully identified gene clusters, 11 were ranked in the top 10 for that disease based on either score of significance and 13 were in the top 20. The X ²a test favours multi-domain proteins whereas disease genes that are single domain proteins have a better chance of being detected with X²b.

### Success of Combined Methods

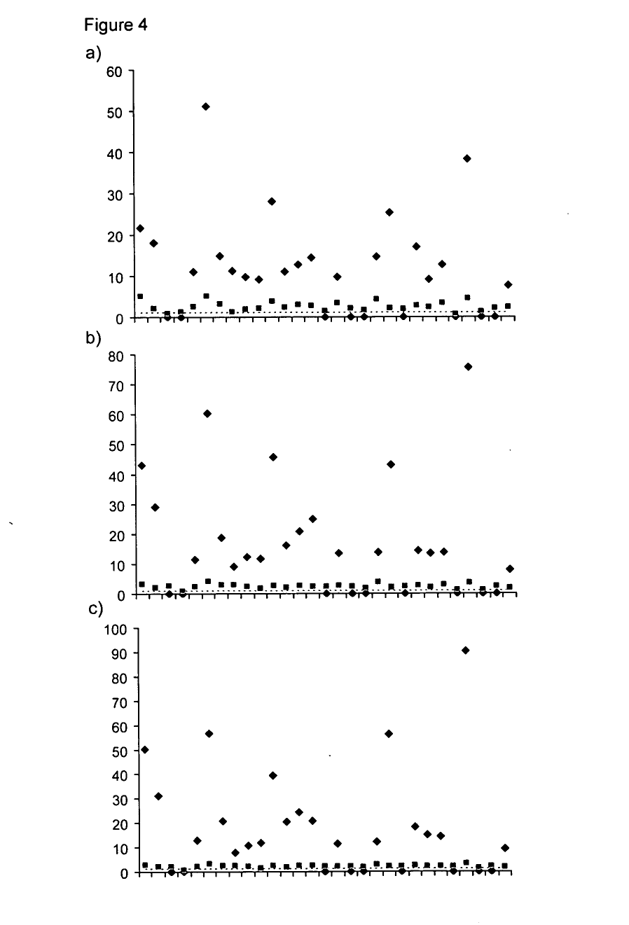

FIG. 4 shows the enrichment scores for each disease using the combined methodology. The combined methods are better than random selection in 20 of the diseases and only worse than random when no correct predictions are made.

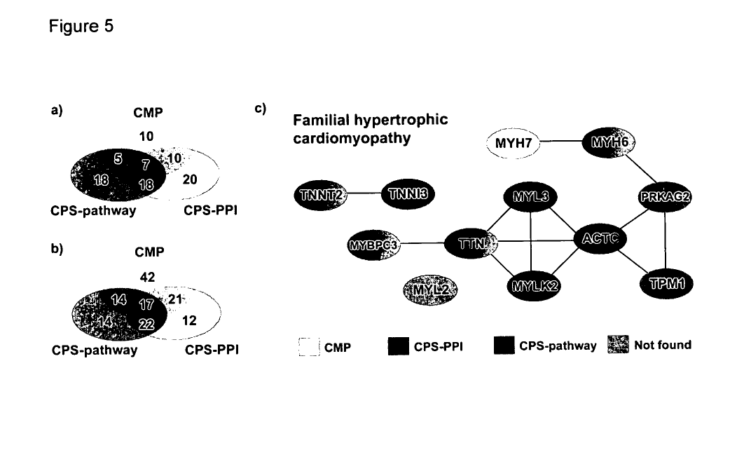

While each method was successful at identifying disease causing genes, performance was improved when combining the methods. The methods tend to be complementary, finding disease genes where the other methods fail: CPS identified disease genes for 10 diseases for which CMP found none and CMP identified nine disease genes that are missed by CPS (FIG. 5).

The probability of finding a disease gene can be increased when combining the results from the two methods: sensitivity increases to 0.512 with a specificity of 0.966 for the 50, 100 and 150-gene intervals. Of the rejected genes, only 0.5% will be disease genes. Overall enrichment is 11-fold in the 50-gene interval and 13-fold in the 100 and 150-gene intervals. Removing the literature-derived PPI data only slightly reduces overall performance: sensitivity is 0.424, selectivity is 0.967 and enrichment is 11-fold at the 100-gene interval. When extending the OPHID interaction data to the second level of interaction, overall sensitivity increases to 0.588, but with a reduction in both specificity, 0.934, and enrichment, eight-fold, for each interval size.

An example of the success of the combined methods can be seen for familial hypertrophic cardiomyopathy (cfh) (FIG. 5c). For the 12 known disease-genes, nine were found by CPS and CMP and a further two were found by the PPI data at a distance of three. Both CPS-PPI data and CMP identify disease genes through relationships between Titin (TTN) and myosin binding protein C (MYBPC3), and between Troponin I type 3 (TNNI3) and troponin T2 (TNNT2). CMP exclusively linked disease genes myosin heavy polypeptide 6 (MYH6) and myosin heavy polypeptide 7 (MYH7). The CPS-pathway-data from KEGG links actin (ACTC), myosin light polypeptide kinase 2 (MYLK2), myosin light polypeptide 3 (MYL3) and titin through the ‘regulation of actin cytoskeleton’ pathway.

For the combined multiple-interval predictions at the 100-gene interval, sensitivity greatly improves to 0.835, however specificity and enrichment to fall to 0.626 and 2.2-fold respectively.

### Drug Discovery Pipeline

Target identification and validation is a crucial first step in developing a drug against a given disease. Only 20-30 new chemical entities are approved as drugs in the US each year and only a quarter of these will act on targets not already hit by an existing drug. There is a real need to identify new targets to treat human disease. The present invention can be expanded into an informatics driven drug-discovery pipeline, which will utilise data from the human genome and disease databases to identify druggable-targets for all diseases.

A target is only of value if it can be related to a disease. This process can take many years as target validation is often a multi-step process involving studies in epidemiology, disease physiology and results from animal models. However, in Mendelian disorders, the inheritance of a mutation in a single gene can be linked directly to a phenotype. There are over 5000 phenotypes with a Mendelian pattern of inheritance, and the gene responsible has been identified in approximately 1200 of these (OMIM). The present invention can be used to identify the disease gene for a further 1500 disease loci for which the disease gene remains undetermined.

In the past, pharmaceutical companies have not studied these diseases, either because the affected protein is not amenable to drug intervention, or more likely, the number of people affected is small and, therefore, drug discovery is not economically viable. Patients with uncommon disorders are often neglected and only receive medications that have come from treatments developed for other more common disorders. However, these neglected diseases may hold the key to therapies that could have multiple uses. A single gene in Mendelian disease may provide insight into complex diseases where the same gene accounts for part of the phenotype. For example, statin therapy was specifically developed to patients with a genomic predisposition to high levels of blood cholesterol, but is equally effective for patients with the same condition but from multiple causes.

### Mapping Diseases to the Human Genome

All disease genes and intervals will be extracted from OMIMs morbidmap (downloadable file), OMIM webpages and the literature. The invention can be used to make predictions for possible disease intervals with unknown disease genes. The minimal requirement for prediction is typically one disease gene or two characterized disease intervals with the same or similar phenotypes.

Benchmarking shows that the invention is already better than published candidate gene prediction systems. Currently our CMP method applies Pfam HMMs to annotate candidate proteins, however, Pfam only has coverage for about 65% of the proteins in the human genome. Domain coverage can be extended by using a combined method of domain prediction and threading. The scooby-domain algorithm (George R A, Lin K and Heringa J (2005) Scooby-domain: prediction of globular domains in protein sequence. Nucleic Acids Res 33, W160-W163) and DOMAINATION methodology (George R A, Heringa J. (2002) Protein domain identification and improved sequence similarity searching using PSI-BLAST. Proteins. 48,672-81) can be applied to identify putative domains in proteins without Pfam annotation. These domains will then be threaded against a database of domains with known structure and function. Each disease will have associated pathways extracted from Biocarta and KEGG as well as interaction data from OPHID. Complete domain (module) annotation, pathway data and interaction data will be used by CMP and CPS to identify disease genes.

### Efficient Target and Drug Identification

Most successful drugs achieve their activity by competing for a binding site on a protein with an endogenous small molecule. For a drug to be effective, it must bind to its molecular target with a reasonable degree of potency as well as having an increased likelihood of oral bioavailability (Lipinski's rule-of-five). These strict physiochemical requirements will limit the type of targets that are druggable. A protein target should favour interactions with drug-like compounds. Proteins lacking these features are unlikely to be amenable to therapeutics. The chance of identifying a good target will be increased by focusing on proteins that are known to bind with successfully commercialized drugs. Information on proteins known to be druggable is freely available from DrugBank (Wishart et al. 2006). Each module in a protein/gene sequence can be assigned a profile that associates drug-binding characteristics. Likely drug-targets in the human genome can be identified through homology searches with the assigned modules in DrugBank. Proteins do not work in isolation: while the disease gene may not be readily druggable, there might be more suitable targets found in its corresponding pathways or interaction partners. For example, inherited mutations in APC, a component of the Wnt pathway, can lead to colon cancer. APC is difficult to target, but compounds that block downstream interactions in this pathway are able to suppress growth of tumors arising from the APC mutations. By using interaction and pathway data from the BioCarta, KEGG and OPHID databases we can identify disease pathways and potential targets.

Potential drugs for both monogenic and complex diseases can be sourced from already available medications, most of which are now off patent, that can be repositioned to new uses. Detailed information related to dosing, in vivo pharmacokinetics and toxicity are already available for these drugs. Our pipeline will identify whether a current drug will be suitable and can potentially lead to immediate phase III clinical trials that can be performed sooner and more economically.

### Target Identification Through Opposing Phenotypes

Most drugs antagonize the gene product producing phenotypes that are analogous to loss-of-function mutations in human disease. Therefore, monogenic human disorders provide an ideal source of drug targets. Because mutations alter the level of activity of gene products, they can be thought of as surrogates for perfectly targeted drugs, to agonize or antagonize the gene product. An example is sulphonylureas. These drugs function antagonistically through the receptor SUR1 complex. Loss-of-function mutations in the genes that encode components of this complex cause the rare genomic disorder persistent hyperinsulinaemic hypoglycaemia of infancy (PHHI). The phenotype of PHHI is directly mimicked by the action of the sulphonylureas. Mutations that cause monogenic disorders have been identified in the genes that encode 12 out of the 43 protein targets of the top-selling 100 drugs in 2003.

Two methods for candidate disease gene prediction have been developed. CPS hypothesizes that novel disease genes reside in the same pathways as those of known disease genes and CMP assumes that novel disease-causing genes that produce the same phenotype as known disease genes are likely to have similar functions. The genes in the genomic interval of interest are then tested for relationships to known disease genes or genes in other disease intervals. Both CPS and CMP can effectively recover known disease genes from a broad array of diseases.

Many previous candidate gene prediction methods have relied on functional annotation, such as GO terms, which can be general or absent. Only 25% of human proteins have manually annotated GO terms. Many more human proteins have predicted annotations, but 35% have no annotation at all. Furthermore, these systems will be biased to well studied and well annotated diseases and may not be useful in the analysis of uncharacterized diseases.

The methods of the present invention are based directly on biological data, and differ from older candidate gene prediction techniques which use blanket systems based on descriptive keywords to cover all aspects of disease. Such methods include POCUS, G2D and SUSPECTS. New systems biology approaches to candidate gene predictions, which are based directly on biological data, mine PPI and pathway databases. Those described by Franke et al. 2006 as well as our own CPS fall into this category. Our CMP method is quite different to any other method previously described, in that it tries to associate particular protein modules with specific diseases. Not only does this technique represent a more powerful way of finding homologs than BLAST searches but it also has the potential to find otherwise unrelated proteins that engage in homophilic interactions (for example through EGF domains) or share a common functional unit but are otherwise unrelated, for example the protein kinase domains found in thyroid carcinoma.

Comparison with other methods is difficult as benchmark datasets are different and some methods merely rank candidates without applying a cut-off. In an attempt to fairly assess our methods compared to others, we have used the disease set as applied in the analysis of POCUS. Turner et al previously compared other methods against POCUS by calculating and comparing enrichment ratios: van Driel et al. studied eight diseases and reduced an average 163 genes to 22, producing a seven-fold enrichment. Freudenberg and Propping found two-thirds of disease genes in the top 15% of candidates, giving a seven-fold enrichment. Generally, these keyword methods have been shown to provide a seven to 10-fold enrichment. The updated G2D method is the most successful of these methods, correctly identifying disease genes for 47% of diseases within their ranked top eight predictions, which is below our performance. Using known disease genes as input, we correctly predicted disease genes for 69% of diseases with an average success rate of one in seven (14%) gene predictions and a 13-fold enrichment.

There are only two other methods, POCUS and PRIORITISER, that attempt the more ambitious task of ab initio predictions in the absence of known disease genes. While POCUS makes very few predictions, for the eight diseases that it does make predictions (28%), the quality of prediction is high with a one in four success rate and 23-fold enrichment. The PRIORITISER method by Franke et al. 2006 correctly identified disease genes for 64% of diseases with a success rate of one in eight predictions and a 2.8-fold enrichment. Our combined methods make correct predictions for all diseases with a 2.2-fold enrichment. Another consideration when comparing these results is the range of pseudo-interval sizes used in the benchmark. POCUS used pseudo-intervals based on keyword densities and sizes ranged from 2 to 19 Mb, which are small and more typical of monogenic diseases. Franke et al. 2006 (used intervals of 50, 100 and 150-genes, but only included those genes that had predicted interactions. Our benchmark pseudo-intervals range from 50 genes (from 1 Mb) to 150 genes (up to 51 Mb). The larger interval sizes are realistic for complex diseases and include all genes.

Our side-by-side use of two prediction systems based directly on independent biological data shows the value of this approach. Several prediction systems were benchmarked against each other using obesity and type 2 diabetes phenotypes. A meta-analysis was then used to choose the best candidates based on consensus. The complementarity of data predicted by our two systems (FIG. 5) show that a consensus method is not always appropriate. Had we used this approach far fewer disease genes would have been found. Clearly the independence of data sources needs to be considered before applying consensus approaches. On the other hand, the type of relationships flagged by CMP is clearly related to pathway data. Pathways may expand by gene duplication and subsequent specialization of the daughters, possibly in association with discrete tissue expression. Similarly, protein complexes consisting of homo-oligomers may differentiate by duplication and specialization of genes encoding similar subunits. If pathway and interaction data were comprehensive then the alternative predictions provided by CMP may not be necessary, but clearly this is not yet the case.

Given that several systems biology approaches have now been published, it is worthwhile examining the caveats associated with these methodologies. CPS with PPI data alone found the majority of disease genes in the benchmark tests. But, some of the interaction data is likely to be dubious, because high-throughput experiments such as yeast two-hybrid and TAP systems will associate proteins that would otherwise never be present in the same cell or subcellular compartment. Furthermore, the various PPIs curated from computational searches of the literature have limited overlap with each other, which may be indicative of a high false positive rate. While there is strong evidence to suggest that PPIs are conserved through evolution, errors in the source data will perpetuate through the databases. These caveats make predicted interactions, such as the Bayesian approach applied by Franke et al., inaccurate. As more evidence for PPIs are collected, the performance of CPS and other similar methods will improve. The results using PPI data alone are already very encouraging: the full OPHID dataset enriches the candidate list by 50-fold, far better than any other reported method.

Finally, although some of the predicted disease genes are not currently known to be involved in the disease, which are counted as false positives in this invention, it is possible that they may be uncharacterized disease-genes. Our methods are also available to identify potential disease genes in user-specified intervals.

A new era of genomics and bioinformatics has permitted a genome-scale perspective of disease and is enabling new technologies to identify disease-causing systems. The present invention should accelerate the disease gene discovery process by gathering and sifting through all knowledge of each candidate gene including its homologues and interaction partners. In addition, it should significantly reduce the cost of expensive experimental studies. Identification of the disease gene enables targeted research on how mutations in the gene contribute to disease and provides specific leads towards cures. The results using the present invention are better than other reported methods for disease gene prediction. Previous methods have relied on functional annotation alone, such as GO terms, which can be general or absent. CPS and CMP utilise information from protein sequence and interaction databases, enabling accurate disease gene identification. In the multiple interval input mode, the present invention does not require a priori knowledge of the disease or disease genes. The present invention should, therefore, be a powerful tool in candidate disease gene prediction for poorly characterised diseases.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A system for profiling a genomic sequence comprising:
assigning modules to a genome, wherein each module has a defined sequence characteristic and the genome is divided into modules;
assigning a value or weight to a module for a given profile, wherein the presence of one or more modules in a genomic sequence contributes to the profile of the genomic sequence relative to its value or weight;
analysing a genomic sequence to identify modules present; and
assigning a profile to the genomic sequence based on the presence of the modules and their respective value or weight.

2. The system according to claim 1 wherein the genomic sequence is an amino acid sequence of a protein and each module is a universal re-occurring unit found in protein sequences.

3. The system according to claim 1 wherein the genome forms the encoding region and the encoding region is divided into different modules.

4. The system according to claim 1 wherein the profile is selected from the group consisting of a gene or loci associated with a phenotype, disease, drug-binding characteristic, trait associated to pharmacogenomics, associated interacting genes, association with a phenotype, associated or interacting modules, and associated biochemical pathways, and associated modules within biochemical pathways or interacting models with profiles with characteristics described here.

5. The system according to claim 4 wherein the phenotype is a disease or a quantitative trait locus (QTL).

6. The system according to claim 4 wherein the profile is an association with a disease.

7. The system according to claim 4 wherein the profile is a drug-binding characteristic.

8. The system according to claim 1 wherein a given value or weight of a module assigned to a profile is obtained by identifying modules associated with a given phenotype (directly or indirectly through pathways or complexes) and assigning a score based on the similarity of a module to modules associated with a specific phenotype.

9. The system according to claim 1 wherein a given value or weight of a module assigned to a profile is obtained by identifying enrichment of those modules in loci (genomic regions) known to be associated with the phenotype.

10. The system according to claim 1 wherein a module is assigned a value or weight according to its presence in sequences associated with the profile.

11. A system for profiling an amino acid sequence to identify an associated profile, the system comprising:
assigning modules to the protein coding region of a genome to divide the genome into modules, wherein each module has a defined amino acid characteristic;
assigning a value or weight to a module for a given profile, wherein the presence of one or more modules in an amino acid sequence contributes to the profile of the sequence relatively to its value or weight;
analysing an amino acid sequence to identify modules present; and
assigning a profile to the amino acid sequence based on the presence of the modules and their respective value or weight.

12. The system according to claim 11 wherein the profile is selected from the group consisting of a gene or loci associated with a phenotype, disease, drug-binding characteristic, trait associated to pharmacogenomics, associated interacting genes, association with a phenotype, associated or interacting modules, and associated biochemical pathways, and associated modules within biochemical pathways or interacting models with profiles with characteristics described here.

13. The system according to claim 12 wherein the phenotype is a disease or a quantitative trait locus (QTL).

14. The system according to claim 12 wherein the profile is an association with a disease.

15. The system according to claim 12 wherein the profile is a drug-binding characteristic.

16. The system according to claim 11 wherein a given value or weight of a module assigned to a profile is obtained by identifying modules associated with a given phenotype (directly or indirectly through pathways or complexes) and assigning a score based on the similarity of a module to modules associated with a specific phenotype.

17. The system according to claim 11 wherein a given value or weight of a module assigned to a profile is obtained by identifying enrichment of those modules in loci (genomic regions) known to be associated with the phenotype.

18. The system according to claim 11 wherein a module is assigned a value or weight according to its presence in sequences associated with the profile.

19. A system in computer readable form containing modules with defined amino acid characteristics wherein each module having an assigned value or weight for one or more profiles.
